(19) United States
(12) Patent Application Publication
(10) Pub. No.: US 2020/0009283 A1
(43) Pub. Date: Jan. 9, 2020

(54) Deodorizing System

(71) Applicants: Joel Gardner, Orlando, FL (US); Denny Bauman, Fort Myers, FL (US)

(72) Inventors: Joel Gardner, Orlando, FL (US); Denny Bauman, Fort Myers, FL (US)

(21) Appl. No.: 16/027,697

(22) Filed: Jul. 5, 2018

(51) Int. Cl. A61L 9/12; A61L 9/04

(52) U.S. Cl. CPC A61L 9/122; A61L 9/046; A61L 2209/212; A61L 2209/12; A61L 2209/111

(57) ABSTRACT

The present invention discloses a system for deodorizing an environment comprising a fan disposed within a housing. The fan creates an air stream by pulling air through an intake and expelling air through an exhaust. An ozone generator is disposed within the housing. As air is moved past the ozone generator, oxygen in the air is converted to ozone and expelled through the exhaust to deodorize the surrounding environment.

### TECHNICAL FIELD

The present invention generally relates to air purifying and deodorizing systems, and more specifically, relates to portable systems for deodorizing a variety of environments.

### BACKGROUND

Ozone is a colorless, pungent gas having powerful antioxidant properties. The molecule is formed naturally whenever oxygen encounters an electric arc, such as lightning or wavelengths of ultraviolet rays. Ozone is comprised of three oxygen atoms which result in a high oxidizing potential. When the molecule contacts pollutants in the air, molecules from the pollutant attach to the unstable oxygen atom on the ozone molecule such that dioxygen levels are restored. The oxidation results in the deodorization of the air where ozone is present.

While ozone occurs naturally, its generation is sporadic and not a regular means for deodorizing an environment. While natural UV light from the sun also contributes to the generation of ozone, the ozone is only created in areas where the UV light can reach. This often leaves gym bags, closets, and other hard to reach areas with an unpleasant odor. To mitigate these odors, deodorizers have been created and are relatively common in the arts as seen in the examples below. However, these solutions do not provide for a system which can be utilized in both large and small environments, and do not employ UV-created ozone to do so.

U.S. Pat. Pub. No. US2005/126033 A1 to David Macher and assigned to THERM-IC Products GmbH discloses a device for drying shoes, gloves, garments or the like which comprises a fan producing a stream of air within a housing. The housing is provided with at least one air exhaust opening which is connected to an air nozzle. Arranged inside the housing or a tube connected with the housing is a closable cavity with a cover in which a carrier provided with essential oils is accommodated, wherein the cover is to be opened towards the air channel. The cavity is connected to the air channel only when the fan is in operation. Also, at least one UV light emitting diode can be arranged in the stream of air whose radiation in association with the oxygen contained in the stream of air produces ozone for disinfection and deodorization.

U.S. Pat. No. 6,447,731 to Shin-Ching Sun and Hubert Chou discloses a cleaning device including a sensor, a circuit board, a fan motor, an optical catalyst glass fiber filter mesh, an anion and ozone generator and a power supply. The cleaning device can collect dust and remove odor. Also, the cleaning device can generate ozone and natural anion to activate human cells and help in blood circulation. Also, utilizing a USB (Universal Serial Bus) signal cable or IBM PS/2 signal cable, the cleaning, device can be connected to a personal computer to serve as peripheral equipment of the computer and form an environmental quality monitoring system. The computer can show the air quality index of the environment. The index can be presented through image or sound of the computer.

U.S. Pat. No. 5,015,442 to Yoichi Hirai and assigned to Tokai Kogyo Co. discloses a sterilizing/deodorizing apparatus having a fan for creating an air flow in one direction in a box-like body. The body has divided air passages, one of the air passages having an ozonizer and an air-permeable ozone-decomposing catalyzer, and the other air passage merely allowing untreated air to flow therethrough.

U.S. Pat. No. 6,508,982 to Masami Shoji and assigned to Kabushiki Kaisha Seisui discloses an air-cleaning apparatus and air-cleaning method whereby air is cleaned with an air flow containing ions and ozone generated by corona exhaust. The apparatus has a wind tunnel means including a trumpet-shaped member having an opening at the bottom thereof and a cylindrical member connected to the opening at the bottom of the trumpet-shaped member. The wind tunnel means has a titanium dioxide metal evaporated onto the surface thereof. A needle electrode is placed in front of the trumpet-shaped member of the wind tunnel part near an axis of the trumpet-shaped member. An annular electrode is formed on the inner surface of the wind tunnel means. A high-voltage generating unit applies voltage between the needle electrode and the annular electrode. A housing accommodates the needle electrode, the annular electrode, and the wind tunnel part and has an air inlet on a side thereof closer to the needle electrode. The housing further has an air outlet on a side thereof closer to the cylindrical member of the wind tunnel part. Corona exhaust is induced by applying a high voltage between the needle and. annular electrodes, thereby generating an air flow containing ions and ozone and thus cleaning the air.

U.S. Pat. Pub. No. US2010/0178196 to Christopher John Garner discloses a bio air sterilization system and method of use that can remove and render benign harmful contaminants and particulates. such as bacteria, viruses, and molds, from air within an enclosed area, as well as, in principle, from the exposed surfaces located within the enclosed area. In one aspect, the sterilization system includes a self-contained, mobile sterilization unit that includes at least an ultraviolet array, an air flow control mechanism for diverting the air flow within the system through either a filter or through an ozone removal zone, an ozone generator, and a blower apparatus to pull the air through the system and out through ports, such as a nozzle system, to the surrounding environment.

U.S. Pat. Pub. No. US2008/0213125 A1 to Nigel Boast discloses a method of sterilizing a closed environment is provided in which an ozone generator is placed into the closed environment. It then generates ozone to a predetermined ozone concentration and increases the humidity of the closed environment. The ozone concentration is maintained at the predetermined ozone concentration for a predetermined period of time, and after the period of time has expired, the ozone is depleted. When the ozone concentration is reduced to a predetermined safe level, the ozone generator signals.

It can be seen that an improvement in the arts related to air purifying and deodorizing systems is needed. The solution should be portable while providing a means for deodorizing and purifying both large and small environments. One such solution is provided in a variety of embodiments herein.

### SUMMARY OF THE INVENTION

This summary is provided to introduce a variety of concepts in a simplified form that is further disclosed in the detailed description of the invention. This summary is not intended to identify key or essential inventive concepts of the claimed subject matter, nor is it intended for determining the scope of the claimed subject matter.

The embodiments herein provide for a deodorizing system which can be utilized to remove unpleasant odors in both confined environments such as a gym bag or drawer, in addition to large environments such as an entire room or apartment. The system uses ultraviolet light to convert oxygen in the environment into ozone, a known deodorizer commonly employed in the arts. The system is comprised of a housing having a first end and a second end. An air intake is positioned at the second end of the housing. A fan forces an air stream through the air intake and expels air through the exhaust at the first end of the device. The ozone generator is positioned within the housing to convert oxygen in the air stream to ozone. Ozone is dispersed throughout the environment, via the fan.

A power source provides electrical energy to the controller. The controller is operable to provide functionality to the device via a processor in communication thereto. The device can include a timer which is in operable communication with the controller to operate the device for a predetermined period of time. The device can also employ a plurality of operational modes including an automatic mode and manual mode of deodorizing the environment.

In one aspect, a user performs the steps of procuring the deodorizing system and disposing the system within the environment. The deodorizing system is activated via the controller, and the user vacates the environment while sealing the system therein. Following a predetermined time period which is generally one hour, the user returns to the environment which has been deodorized.

In a preferred embodiment, the UV light ozone generator emits a wavelength at 253.7 nm. This wavelength is known as a preferable wavelength for converting oxygen into ozone.

In one aspect, the device is comprised of a plurality of sensors to facilitate a manual operation mode. Sensors may include ozone sensors, temperature sensors, pressure sensors, light sensors, motion sensors, rheostats, and similar useful implements. Each sensor sends inputs to the controller which can determine if the device is operating or on standby.

In one aspect, the fan has a variable speed which can be modified by the controller. The preferred speed of the fan can be determined by the size of the environment, as well as the period of time during which a deodorization cycle occurs.

Moreover, in accordance with a preferred embodiment of the present invention, other aspects, advantages, and novel features of the present invention will become apparent from the following detailed description in conjunction with the drawings.

### BRIEF DESCRIPTION OF THE DRAWINGS

A more complete understanding of the present invention and the advantages and features thereof will be more readily understood by reference to the following detailed description when considered in conjunction with the accompanying drawings wherein:

FIG. 1 illustrates a perspective view of the deodorizing system housing and power source, according to an embodiment of the present invention;

FIG. 2 illustrates a perspective view of the deodorizing system, according to an embodiment of the present invention;

FIG. 3 illustrates a perspective view of the deodorizing system and internal components thereof, according to an embodiment of the present invention;

FIG. 4 illustrates a top plan view of the deodorizing system disposed within a gym bag environment; according to an embodiment of the present invention;

FIG. 5 illustrates a top plan view of the deodorizing system disposed within an enclosed gym bag environment; according to an embodiment of the present invention; and

FIG. 6 illustrates a block diagram of the controller system, according to an embodiment of the present invention.

### DETAILED DESCRIPTION

The specific details of the single embodiment or variety of embodiments described herein are to the described system and methods of use. Any specific details of the embodiments are used for demonstration purposes only and not unnecessary limitations or inferences are to be understood therefrom.

Any reference to “invention” within this document is a reference to an embodiment of a family of inventions, with no single embodiment including features that are necessarily included in all embodiments, unless otherwise stated. Furthermore, although there may be references to “advantage's” provided by some embodiments, other embodiments may not include those same advantages, or may include different advantages. Any advantages described herein are not to be construed as limiting to any of the claims.

Before describing in detail exemplary embodiments, it is noted that the embodiments reside primarily in combinations of components related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second” and the like may be used solely to distinguish one entity or element from another entity or element without necessarily requiring or implying any physical or logical relationship or order between such entities or elements.

In general, the embodiments herein provide for a deodorizing system which can be used in both large and small environments. For clarity, a large environment can include but is not limited to a domicile or room thereof, a commercial area, garage, automobile, boat, or similar environment. A small environment can include but is not limited to a gym bag, backpack, closet, cabinet, storage space, or similar space in an environment. The invention is useful in removing odors from the environment. Sources of odors which can be eliminated include but are not limited to bacteria, mold, mildew, smoke, formaldehyde, infectious agents, pet odors, among other sources of odors.

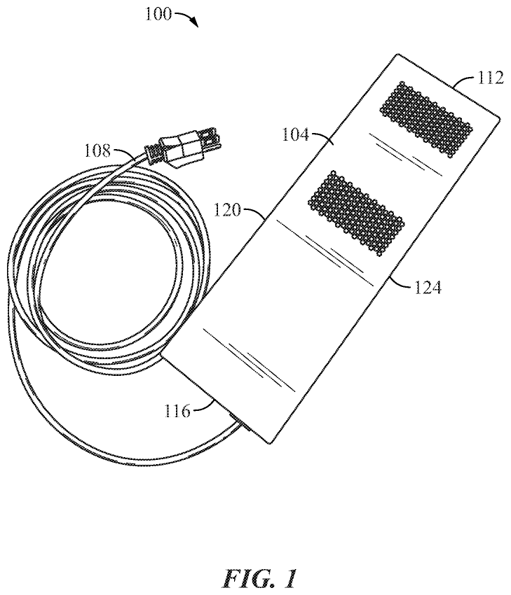

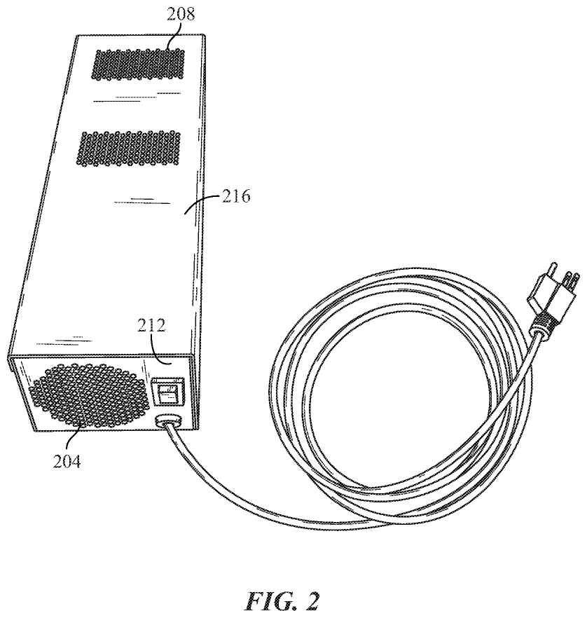

In reference to FIG. 1 and FIG. 2, the device 100 is illustrated having a housing 104 having a first end 112, a second end 116, left and right sides 120, 124, and surface 216. A power source 108 supplies electrical energy to the internal components of the device as described herein. The device 100 can operate using an AC outlet, or alternate embodiments can allow for a battery or similar energy storage device to be integrated into the housing 104 to supply power.

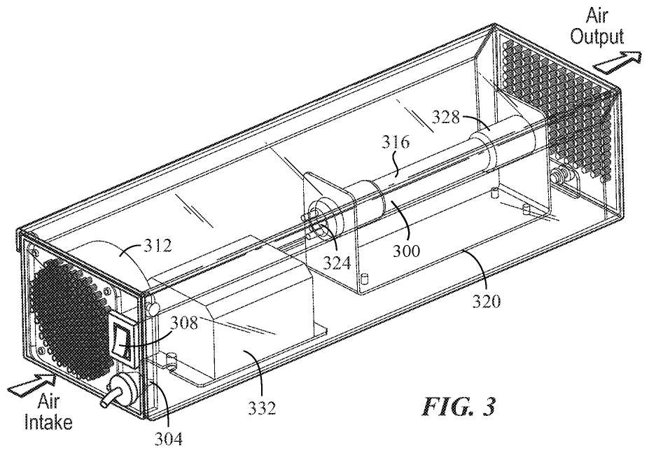

In a preferred embodiment, an air intake is positioned at the second end 116. The intake 204 is configured as a plurality of openings on the surface 212 such that air can flow into the interior cavity of the housing 104. At least one air exhaust 208 portion is positioned on the housing. The exhaust 208 can be positioned on the upper surface 216 (as seen in FIG. 1 and FIG. 2) or on the surface of the first end 112 as seen in FIG. 3. The exhaust 208 is configured as a plurality of openings to permit the egress of air (including ozone) therethrough.

The housing 104 and surfaces thereof can be releasably engaged with one another to permit cleaning, replacement, or servicing of each component therein. Each component forms a sealed engagement to ensure proper air flow through the housing via the air intake 204 and exhaust 208.

In an embodiment, at least one filter mechanism is provided at the air intake such that air entering the housing 104 is clean of large airborne particles. Preferentially, the filter mechanism is easily accessed for removal, replacement, and cleaning without opening the housing 104. This can be accomplished via an aperture positioned on the exterior of the housing 104. The filter can then be removably engaged with the housing 104.

In reference to FIG. 3, a representation of the device 100 and inner components disposed within the housing 104 are illustrated. To generate ozone, the known means of using ultraviolet radiation is employed. Means for ozone conversion includes an ozone generator 300, and specifically an ultraviolet (UV) light ozone generator. Other means known in the arts include a corona discharge generator. The UV generator 300 is known to have stable ozone output levels throughout a variety of humidity ranges and can therefore be used in a wide range of environments when compared with the corona discharge generator (which can have significantly reduced output in environments with moderate humidity).

In one embodiment, a suitable lamp 316 is a germicidal lamp provided by Light Sources, Inc. The lamp 316 includes a base face to opposite pin length of 135 mm, a base face to base face length of 127 mm, and a diameter of 15 mm. The lamp 316 is configured to perform using a lamp wattage of 10 W, a lamp current of 425 mA, and lamp voltage of 27 V. To produce ozone, specific UV outputs are required. Typically, a high out is at about 185 nm, and low output is at about 254 nm. In the present example, the output is configured at 253.7 nm at 2 W. The intensity at 1 m is 28 μW/cm². The average lifespan of the lamp 316 is 16,000 hours at standard operational capacity. The lamp 316 is comprised of a four-pin base 324, 328 at each end.

It is understood that a variety of suitable lamps are available in the arts. These can be implemented in the present invention to achieve suitable ozone output for deodorization of a variety of environments, both small and large.

A fan 312 is positioned near the air intake 204 to generate an air stream through the openings of the air intake 204 to the exhaust 208. The lamp 316 is positioned along the travel path of the air stream to convert oxygen to ozone. In this manner, oxygen enters the intake 204, the oxygen is converted to ozone within the interior of the housing 104 via the lamp 316, and ozone is expelled through the exhaust 208 portions.

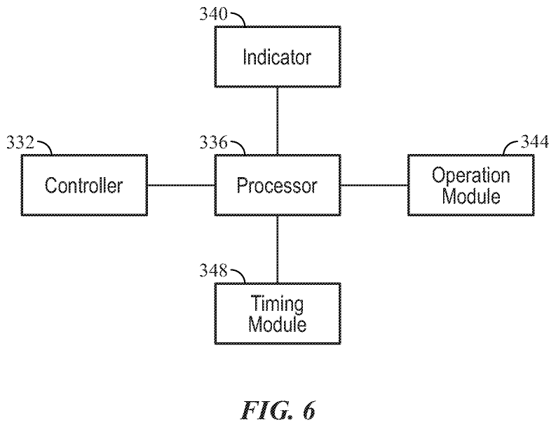

In the preferred embodiment, a controller 332 is disposed within the housing 104. The controller is in electrical communication with the power source 108 and provides functionality to the device 100. In reference to FIG. 6, the controller 332 is comprised of a processor 336. The processor 336 is configured to instruct a plurality of modules including an operation module 344 and a timing module 348. The operation module 344 may be in communication with a memory component wherein operation settings are stored relating to lamp settings and operational time settings. The timing module 348 is configured to time the length at which the device 100 is operating to produce ozone and effectively deodorize the environment.

In an embodiment, an indicator 340 is positioned on the exterior of the device to alert the user of the operation or function of the device 100 in real-time. In one example, the indicator is an LED light having a plurality of color settings to indicate an operations mode or status. The indicator showing the color red can indicate the device 100 is OFF and therefore not producing ozone, while a green light can indicate the device 100 is on and producing ozone. The controller 332 may also modulate the lamp 316 and output thereof.

The controller 332 may be coupled with a transceiver to send and receive instructions, data, or likewise information to and from a smart device. In this manner, a user can send instructions to the controller 332 to turn the device to an ON or OFF operation, as well as determine operational settings of the device 100.

The device 100 can be operating using a switch 308 such as an ON/OFF switch. In further embodiments, the device can be comprised of an interface, allowing the user to select from a variety of operational functions thereon.

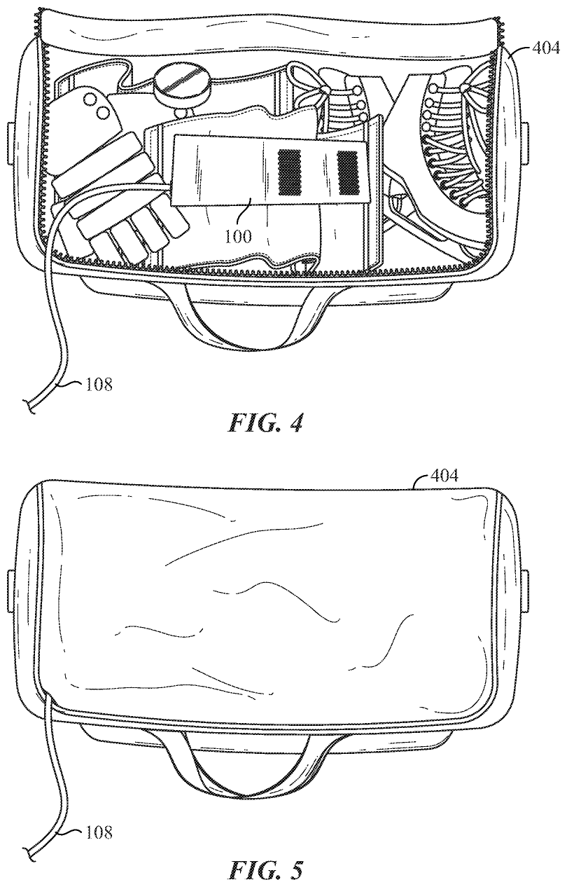

Now referring to FIG. 4 and FIG. 5 the device 100 is disposed in an environment 404. In the illustrated embodiment, the device 100 is disposed within a bag (e.g., a gym bag). The housing 104 is dimensioned to be small enough to be contained within a gym bag, backpack, closet, cabinet, drawer, or other small and confined space function as a deodorizer thereto. The device 100 and specifically the fan 312 and lamp 316 is configured to be capable of deodorizing a large space such as an entire room. In use, the user selects an environment (small or large) that needs to be deodorized. The user then disposes the entire device 100 within the environment and seals the environment to enclose the device 100 therein. In the example provided in FIG. 4 and FIG. 5, the device is placed in the gym bag environment 404. The gym bag can be empty or include materials, such as clothes or other equipment. The power supply 108 can extend out of the environment 404 to engage with an outlet if necessary. In an embodiment, wherein a room or larger environment having an outlet is deodorized, the outlet is not required to be exterior to the environment. The user then turns the device to an ON setting, utilizing switch 308) and vacates the environment. In the example of the gym bag environment 404, the user seals the gym bag and vacates the area surrounding the gym bag. It may be preferable to leave the gym bag in an enclosed and vacated area during the deodorization process.

In an embodiment, a memory stores a plurality of operating modes which are selectable by the user. The device 100 can operate in a manual mode, wherein the user manually turns the device 100 to ON to deodorize the environment. The device 100 can also operate in a manual mode wherein the device can operate independently of user input. This can be useful in an environment that undergoes prolonged periods of vacancy such as a car, boat, or closet. Manual operation can be facilitated by a plurality of sensors including but not limited to any of the following: a motion sensor, an ambient light sensor, a rheostat, a pressure sensor, a moisture sensor, a temperature sensor, or any other useful environmental sensing means. An ozone sensor can be incorporated such that the device only operates when ozone is within a predetermined range in the environment.

A rheostat can be electrically connected to the generator 300 to allow for the adjustment of ozone output or concentration. A programmable timer/processor 336 could be incorporated to operate the generator 300 automatically. A switch 308 is disposed on the exterior of the housing 104 to allow for a deodorizing cycle to be started when the switch 308 is depressed.

An ozone detector may be incorporated as a safety measure. It would measure ambient air surrounding the ozone unit and adjust or cease production of ozone if ambient levels became too high.

Various forms of a fan 312 may be used. Conveniently there is an electric fan running at a speed of the order of 1000 to 4000 rpm. A range of different flow rates may conveniently be obtained for a given fan speed by merely changing the fan blade angle. Modulation of the fan 312 can permit the device 100 to suitably deodorize a wide range of environments.

Electrostatic filters are particularly preferred and are well-known in the art. In principle, they use charged filter media to trap charged particles. Most small units are passive in that they use the friction due to the passage of air through the filter to generate a static charge on specialized materials, which is the principle utilized in the well-known HEPA filters. More recently, permanently polarized electric filter media with particularly high electrostatic charge surfaces, as described in (Myers & Arnold, Winter 2003, International Nonwovens Journal and International patent application publication WO 00/01737), have formed the basis of so-called HAF (High Air-Flow) filters, which have far greater face speeds while maintaining highly efficient filtering. Large industrial electrostatic precipitators (or ‘electronic’ filters) use charged plates or a corona exhaust to actively impart a charge to airborne particles. As used herein, ‘electrostatic filters’ includes all of these types.

An electrostatic post-filter may provide a synergistic benefit with the filter materials, which in some way increases the electrostatic attraction between the airborne particulates emerging from the inactivation zone and the filter surfaces, which results in a significant reduction in the size of the particles which may be trapped by the electrostatic filter used in preferred forms of the invention. This is particularly significant concerning viruses as these generally have a size of the order of 0.1 micron, which is below the normal minimum particle size of 0.3 microns which can be trapped by HAF filters. Examination of HAF filters used in apparatus of the present invention indicates though that even such viral particles can be successfully trapped.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A deodorizing system comprising:
a. a housing having a first end and a second end;
b. at least one air intake positioned at the second end and at least one exhaust positioned at the first end;
c. a fan disposed in the housing, wherein the fan is configured to provide a stream of air such that air ingresses through the at least one intake, and egresses through the at least one exhaust;
d. at least one ozone generator positioned within the housing, wherein the stream of air flows passes the ultraviolet ozone generator to oxidize dioxygen molecules present in the stream of air;
e. a power source, driving the fan, the power source electrically connected to a controller.

2. The system of claim 1, wherein the ozone generator is an ultraviolet light ozone generator.

3. The system of claim 2, further comprising a lamp mounted to the housing, wherein the lamp is in operable communication with the controller.

4. The system of claim 3, wherein the lamp is an ultraviolet light configured to emit wavelengths at about 254 nm.

5. The system of claim 1, further comprising an indicator operatively connected to the controller, wherein the indicator alerts the user.

6. The system of claim 1, further comprising a timer operably connected to the fan and the ozone generator, wherein the timer activates the fan and the lamp for a predetermined period of time.

7. The system of claim 1, further comprising an operation module in operable communication with the controller, wherein the operation module outputs an operation mode determined by the user.

8. A deodorizing system comprising:
a. a housing having a first end and a second end;
b. at least one air intake positioned at the second end and at least one exhaust positioned at the first end;
c. a fan disposed in the housing, wherein the fan is configured to provide a stream of air such that air ingresses through the at least one intake, and egresses through the at least one exhaust;
d. at least one ultraviolet ozone generator positioned within the housing, wherein the stream of air flows passes the ultraviolet ozone generator to oxidize dioxygen molecules present in the stream of air;
e. a power source, driving the fan, the power source electrically connected to a controller.

9. The system of claim 8, wherein the housing is cuboid including a top surface and a bottom surface.

10. The system of claim 9, wherein the at least one exhaust is positioned on the top surface.

11. The system of claim 8, further comprising a timer operably connected to the fan and the lamp, wherein the timer activates the fan and the lamp for a predetermined period of time.

12. The system of claim 8, further comprising an operation module in communication with the controller, wherein the operation module determines an appropriate operation mode.

13. The system of claim 8, wherein a user performs the steps of:
a. procuring the deodorizing system;
b. disposing the system in an environment;
c. activating the deodorizing system via the controller;
d. sealing the deodorizing system in the environment for a predetermined time period, wherein the sealed environment is uninhabited; and
e. opening the sealed environment following the predetermined time period

14. The system of claim 8, further comprising an indicator operatively connected to the controller, wherein the indicator alerts the user.

15. A deodorizing system comprising:
a. a housing having a first end and a second end;
b. at least one air intake positioned at the second end and at least one exhaust positioned at the first end;
c. a fan disposed in the housing, wherein the fan is configured to provide a stream of air such that air ingresses through the at least one intake, and egresses through the at least on exhaust;
d. at least one deodorizing element positioned within the housing, wherein the stream of air sufficiently contacts the stream of air to oxidize dioxygen molecules present in the stream of air; and
e. a power source, driving the fan, the power source electrically connected to a controller,
wherein a user performs the steps of:
f. procuring the deodorizing system;
g. disposing the system in an environment;
h. activating the deodorizing system via the controller;
i. sealing the deodorizing system in the environment for a predetermined time period, wherein the sealed environment is uninhabited; and
j. opening the sealed environment following the predetermined period of time.

16. The system of claim 15, wherein the lamp is an ultraviolet light configured to emit wavelengths at about 254 nm.

17. The system of claim 15, further comprising a rheostat in electrical communication with the controller for adjusting the output of the lamp.

18. The system of claim 15, wherein the rheostat is in electrical communication with the fan to regulate the fan.

19. The system of claim 15, wherein the deodorizing device is automated via a plurality of sensors positioned on the device.

20. The system of claim 19, wherein the plurality of sensors includes any combination of the following: a timer, a pressure sensor, an ozone sensor, a motion sensor, a light sensor, and a temperature sensor.
